(19) United States
(12) Patent Application Publication
(10) Pub. No.: US 2002/0169560 A1
(43) Pub. Date: Nov. 14, 2002

(54) Analysis mechanism for genetic data

(75) Inventors: Evangelos Hytopoulos, San Mateo, CA (US); Brett Miller, Albany, CA (US); Sandip Ray, San Francisco, CA (US)

Correspondence Address: LAW OFFICES OF JAMES D. IVEY, 3025 TOTTERDELL STREET, OAKLAND, CA, 94611-1742, US

(73) Assignee: X-Mine

(21) Appl. No.: 09/854,427

(22) Filed: May 12, 2001

(51) Int. Cl. G06F 19/00; G01N 33/48; G01N 33/50

(52) U.S. Cl. 702/19; 702/20

(57) ABSTRACT

Results of statistical clustering and/or correlation analysis of genetic or proteomic expression data such as microarrays, gene chips, or protein chips are used, e.g., as response variables, in further analysis of expression data. In particular, an array of expression data is clustered using a cluster tool to produce an array of expression clusters. Each of the expression clusters represents the same experiments represented by the original expression array. Accordingly, each cluster of the array is of the proper form to be used as a response variable of expression values. Using an expression cluster as a response variable for either supervising clustering or correlation analysis allows correlation between such an expression cluster and other expression data.

### CROSS-REFERENCE TO RELATED APPLICATIONS

The present application is related to the following co-pending patent applications which are filed on the same date on which the present application is filed and which are incorporated herein in their entirety by reference: (i) patent application Ser. No. 09/______ entitled “Analysis Mechanism for Genetic Data” by Evangelos Hytopoulos, Brett Miller, and Sandip Ray (Attorney Docket P-2172D2) and (ii) patent application Ser. No. 09/______ entitled “Web-Based Genetic Research Engine” by Evangelos Hytopoulos, Brett Miller, and Sandip Ray (Attorney Docket XMNE:0101).

### FIELD OF THE INVENTION

The invention relates to computer-implemented analysis of genetic data and, in particular, a mechanism for improved correlation and clustering analysis of genetic data.

### BACKGROUND OF THE INVENTION

The human genome has recently been mapped, and the map of the human genome is widely distributed for all to see. However, while we are able to point to the location of any human gene within the 23 chromosomes that make up the human genome, we still do not know what aspect of human biology each gene affects. Thus, the mapping of the human genome can be thought of as merely the first step in benefitting from understanding the genetic composition of human beings. The second step is determining what effect each gene, or various combinations of genes, have on human biology. Turning that second step on its head, the new quest is to determine what genes affect a particular human ailment.

To answer this latter question, genetic data is collected from people having various health states—from normal to various states of ailments of interest. Currently, various types of cancer are predominant areas of intense focus in the medical research community and genetic samples are taken from patients having various stages of various types of cancer. The amount of genetic data collected is quite large, due to both including many samples of genetic data and the sheer size of the fully represented genome for each sample. Accordingly, such genetic data is collected in DNA microarrays, which are sometimes commonly referred to as biochips, DNA chips, gene arrays, gene chips, and genome chips.

DNA microarrays exploit a phenomenon known as base-pairing or hybridization. In particular, in DNA, adenine (commonly referred to as “A” in the context of genes) with thymine (“T” in genetic context) tend to pair with one another, and guanine (“G” in genetic context) and cytosine (“C” in genetic context) tend to pair with one another. In RNA, A and uracil (“U” in genetic context) tend to pair with one another, and G and C tend to pair with one another.

To form the array, genetic samples are arranged in an orderly manner (typically in a rectangular grid) on a substrate. Examples of commonly used substrates includes microplates and blotting membranes. The samples can be laid by hand or by robotics. Samples range in size from less than 200 microns in diameter to over 300 microns in diameter. More modern microarrays include an array of oligonucleotide (20˜80-mer oligos) or peptide nucleic acid (PNA) probes, and the array is synthesized either in situ (on-chip) or by conventional synthesis followed by on-chip immobilization. The array on the chip is exposed to labeled sample DNA, hybridized, and the identity/abundance of complementary sequences are determined. Sometimes referred to as DNA chips, this process is included in the term DNA microarrays as used herein.

DNA microarrays are fabricated by high-speed robotics, generally on glass or nylon substrates. A probe is applied to the entire array simultaneously. As used herein, a probe is a substance applied to an array for testing purposes. One example of a probe is a tethered nucleic acid with a known sequence. On the other hand, a target as used herein is a free nucleic acid sample whose identity or abundance is being detected in the array. Application of the probe to the entire array allows determination of complementary binding, thus allowing massively parallel gene expression and gene discovery studies. An experiment with a single DNA chip can provide researchers information on thousands of genes simultaneously. This represents a dramatic increase in throughput such that analysis of genetic data is becoming increasingly practical for more and more human conditions.

There are two major uses of DNA microarray technology. The first involves identification of the gene sequence. The second involves determination of expression level of genes, generally referred to as the abundance of the genes. In particular, expression or abundance of a gene is a measure of a relative level of activity of the gene in replication or translation in the presence of the probe. By analyzing the abundance of various genes in people of various conditions, a relationship between the genetic state of a person, in terms of relative levels of activity of various genes of that person, and that person's condition is assessed. To conduct such analysis, such arrays of expression levels include metadata describing characteristics of the people whose genetic material is sampled and additional metadata which identifies specific genes whose expression levels are represented in such arrays.

What is needed is a particularly effective mechanism for analyzing DNA array data to determine which genes or combinations of genes are correlated to various human conditions.

### SUMMARY OF THE INVENTION

In accordance with the present invention, results of statistical clustering and/or correlation analysis of genetic or proteomic expression data are used, e.g., as response variables, in further analysis of genetic expression data. In particular, an array of expression data is clustered using a cluster tool to produce an array of expression clusters. As used herein, an expression cluster is a collection of expression data which resembles a single gene or protein resulting from the combination of member genes or proteins of the cluster. One example of such an expression cluster is a weighted average of the members of a particular cluster. It should be noted that each of the expression clusters represents the same experiments represented by the original expression array. Accordingly, each expression cluster of the array is of the proper form to be used as a response variable of expression values. Using an expression cluster as a response variable for either supervising clustering or correlation analysis allows correlation between such an expression cluster and other expression data.

To facilitate such use of clustering results in subsequent processing, resulting cluster arrays are included with unclustered expression data arrays as expression data which a user can select for processing by any of a number of cluster tool and/or any of a number of correlation tools. In addition, the user can specify response variables for supervised cluster tools and for correlation tools. Alternatively, the user can select one or more clusters or expression data from an unclustered expression array for use as such a response variable. In addition, the user is provided with an interface by which the user can select which of a number of cluster tools and/or correlation tools processes the selected expression array.

The extensive user-configurability of the system according to the present invention allows for many different types of analysis of genetic and/or proteomic data in ways heretofore unimagined. For example, the user can specify that a cluster tool form expression clusters from an array of expression data and then specify that the expression clusters themselves are clustered, e.g., using the same or a different cluster tool, to produce clusters of clusters.

### BRIEF DESCRIPTION OF THE DRAWINGS

FIG. 1 is a block diagram of a genetic/proteomic expression data analysis mechanism according to the present invention.

FIG. 2 is a block diagram of the cluster tool of FIG. 1 in greater detail.

FIG. 3 is a block diagram of the correlation tool of FIG. 1 in greater detail.

FIG. 4 is a logic flow diagram illustrating expression data analysis according to the system of FIG. 1.

FIG. 5A is a logic flow diagram illustrating expression data analysis according to the system of FIG. 1.

FIG. 5B is a block diagram summarizing processing according to the logic flow diagram of FIG. 5A.

FIG. 6A is a logic flow diagram illustrating expression data analysis according to the system of FIG. 1.

FIG. 6B is a block diagram summarizing processing according to the logic flow diagram of FIG. 6A.

FIG. 7A is a logic flow diagram illustrating expression data analysis according to the system of FIG. 1.

FIG. 7B is a block diagram summarizing processing according to the logic flow diagram of FIG. 7A.

FIG. 8A is a logic flow diagram illustrating expression data analysis according to the system of FIG. 1.

FIG. 8B is a block diagram summarizing processing according to the logic flow diagram of FIG. 8A.

FIG. 9A is a logic flow diagram illustrating expression data analysis according to the system of FIG. 1.

FIG. 9B is a block diagram summarizing processing according to the logic flow diagram of FIG. 9A.

FIG. 10 is a block diagram of an expression array processed by the system of FIG. 1 according to the present invention.

FIG. 11 is a block diagram of a cluster array processed by the system of FIG. 1 according to the present invention.

FIG. 12 is a block diagram of a supervising array used by the system of FIG. 1.

FIG. 13 is a logic flow diagram of a visual correlation display of expression displays.

FIG. 14 is a block diagram of multiple expression displays in accordance with the present invention.

FIGS. 15, 16, and 17 are respective displays of FIG. 14 shown in greater detail.

### DETAILED DESCRIPTION

In accordance with the present invention, an expression data array processing system 100 statistically analyzes selected ones of expression data arrays 102 using results of previous statistical analysis for guidance. System 100 leverages from the realization that expression data arrays, cluster arrays, and response variables have similar structures and understanding of such similarity facilitates understanding and appreciation of the advantages of system 100.

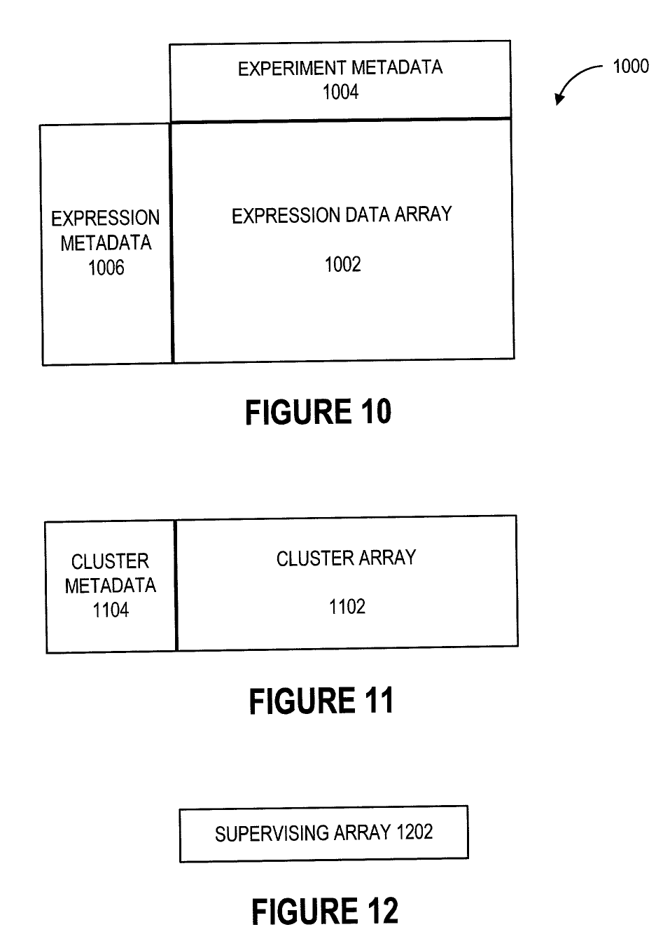

FIG. 10 shows a genetic dataset 1000 which includes an expression data array 1002, experiment metadata 1004, and expression metadata 1006. Expression data array 1002 is a collection of genetic data using gene array technology such as that described above. While such genetic data can have any of a variety of structures when stored on a computer-readable memory, expression data array 1002 is shown and described herein as a two-dimensional array in which each column represents an experiment, e.g., gene expression levels for a particular subject, and each row represents a particular gene, e.g., expression levels for that particular gene for all subjects of expression data array 1002.

While genetic data is described herein with respect to FIG. 10, it should be appreciated that proteomic data, collected using protein chips in a known, conventional manner similar to that described above with respect to gene chip technology, can also be processed and analyzed by system 100 in the manner described herein. When processing proteomic data, each element of array 1002 specifies relative levels of abundance of a particular protein rather than relative levels of abundance of material specific to a particular gene. However, the level of abundance of a protein can be represented in the same manner, e.g., as a degree of expression, and is therefore equally accurately described as expression data herein.

Experiment metadata 1004 stores data representing various conditions of the subjects from which each genetic sample was taken. For example, experiment metadata 1004 can indicate that a particular column of expression data array 1002 represents a genetic sample of a female patient who was 43 years of age and who had a particularly advanced stage of ovarian cancer. Experiment metadata 1004 can specify generally any potentially relevant data for subjects of expression data array 1002 including, for example, demographic data, dates of collection of genetic samples, types of genetic samples, location of sample collection, survival time, expression data from other datasets, etc. Experiment metadata 1004 can store such information directly or indirectly, e.g., by including references to such data stored elsewhere.

In some datasets, each column of expression data array 1002 and experiment metadata 1004 pertains to a distinct subject. In other datasets, multiple columns of expression data array 1002 and experiment metadata 1004 can pertain to the same subject, e.g., to multiple samples taken from the same subject over time. In such datasets, experiment metadata 1004 includes data specifying a time at which each sample is taken. Since genetic expression data represents relative degrees of activity of various genes, such genetic expression data can fluctuate over time and measuring such fluctuations against changes in the subject's condition can be helpful in determining a function of a particular gene. Similarly, proteomic expression data can fluctuate over time and correlating such fluctuations to those of a condition measured over time can help determine a relationship between various protein levels and human conditions.

Expression metadata 1006 stores data identifying the particular genes or proteins represented in respective rows of expression data array 1002. Such identifying data can include, for example, the name, accession number, functional category, brief description, and/or any known associated disorders of the specific genes. Functional categories of genes can include such categories as cell cycle/proliferation/survival, cell surface markers/cell adhesion, cellular metabolism, channel proteins, cytoskeleton, DNA replication/repair, extracellular matrix, kinases/phosphatases, neuronal, protein processing/trafficking, proteolysis, RNA processing, serum/blood cell proteins, signaling molecules/growth factors/receptors, transcription/nuclear proteins, and translation/protein synthesis, for example. Similarly, if data array 1002 represents proteomic expression data, metadata 1006 stores similar data identifying the particular protein represented by the corresponding row of data array 1002.

Thus, expression levels for any genes represented in expression data array 1002 can be located by knowing the particular types of experiments that are of interest and the particular gene. For example, expression levels of a particular gene for all male subjects of a particular range of ages have a particular condition can be located by finding the intersection of that particular gene, located using expression metadata 1006, and experiments matching that particular demographic profile, located using experiment metadata 1004.

In this illustrative embodiment, expression data array 1002, experiment metadata 1004, and expression metadata 1006 are stored separately for efficient access.

Cluster array 1102 (FIG. 11) represents clusters of expression array data. In this illustrative example, cluster array 1102 represents clusters of rows of expression data array 1002 (FIG. 10). Of course, cluster array 1102 can have any of a number of data structures when stored within a computer-readable memory, but is described herein and shown for simplicity and illustration purposes to be a two-dimensional array of expression levels. Each row of cluster array 1102 represents a combination of one or more rows of expression data array 1002 (FIG. 10). For example, the combination can be a weighted average of a number of rows of expression data array 1002. The resulting cluster expression data is a single row of expression data of generally the form of expression data from which the clusters are formed.

Cluster metadata 1104 (FIG. 11) specifies, for each row of cluster array 1102, which rows of expression data array 1002 (FIG. 10) are represented in the row and how the rows of expression data array 1002 are combined. For example, if a particular row of cluster array 1102 (FIG. 11) represents a weighted average of three (3) rows of expression data array 1002 (FIG. 10), cluster metadata 1104 (FIG. 11) identifies the three (3) rows of expression data array 1002 and specifies the weight applied to each of the three (3) rows in forming the weighted average expression data of the cluster. Rows of cluster array 1102 can also represent clusters of rows of experiment metadata 1004 and/or clusters of both metadata and genetic expression data from both experiment metadata 1004 and expression data array 1002.

Cluster array 1102 has the same number of columns as does expression data array 1002. In fact, rows of expression data array 1002 are combined to form rows of cluster array 1102 in such a manner that columns of cluster array 1102 correspond to similarly positioned columns of expression data array 1002. Accordingly, experiment metadata 1004 (FIG. 10) is equally applicable to columns of cluster array 1102 (FIG. 11) to describe demographic and other relevant data pertaining to specific columns of cluster array 1102.

Supervising array 1202 (FIG. 12) can be used as a response variable for supervised clustering tools and for correlation tools as described more completely below. While supervising array 1202 can be organized according any of a variety of data structures, supervising array 1202 is described herein and shown for illustration purposes as an array having the same number of experiments and in positions analogous to experiments of expression data array 1002. Accordingly, experiment metadata 1004 is equally applicable to supervising array 1202 in the manner described above with respect to cluster array 1102.

For each column of supervising array 1202 (FIG. 12), an element specifies an expression value of interest in any of a number of ways. Four (4) such ways are described herein; however, other ways of specifying a gene expression value of interest can be used as well. The four (4) ways in which gene expression values of interest are specified in this illustrative embodiment include: (i) the expression value of interest itself, (ii) a class label specifying a class represented in experiment metadata 1004; (iii) survival time of the subject of each experiment as represented in experiment metadata 1004; and (iv) time series values, e.g., conditions mapped against time. An example of the last way can include, for example, blood pressure measurements taking at respective relative times.

Supervising array 1202, in the form of interesting expression values, can be thought of as expression levels for a single gene—either obtained experimentally or constructed hypothetically in a manner described more completely below. In particular, supervising array 1202 contains one expression level for each column of expression data array 1002.

For class labels, supervising array 1202 includes a class label for each column of experiment metadata 1004. Each class label represents a class of subject from which genetic samples were taken. For example, one class might represent patients with breast cancer while another class represents patients with ovarian cancer and a third class can represent patients with no cancer at all.

For survival times, supervising array 1202 includes a survival time for each subject of each column of experiment metadata 1004. Survival time includes a time, e.g., from some reference time such as first diagnosis or birth for example, and a censor flag. The censor flag indicates whether (i) the subject died at the specified survival time or (ii) the subject lived at least the amount of time specified as the survival time and no further information is available.

For time series, supervising array 1202 includes measured conditions and associated respective times of measurement. The measured condition can be generally any measurable condition of the subjects of experiment metadata 1004 including, for example, blood pressure, heart rate, and blood levels of such things as sugar and other chemicals and various types of cells. The associated times can be relative to some reference time and therefore include time of day, time since diagnosis, time since waking, time since eating, and time since administering a drug, for example. It is possible that the times of measurements specified in supervising array 1202 does not directly match times of expression levels represented in expression data array 1002. In such circumstances, measured conditions for times represented in expression data array 1002 are interpolated and/or extrapolated from measured conditions specified in supervising array 1202 (FIG. 12) using conventional techniques.

Thus, expression data array 1002, cluster array 1102, and supervising array 1202 all represent the same number of experiments and are accurately described by experiment metadata 1004. Such is true if cluster array 1102 and supervising array 1202 correspond to expression data array 1002, e.g., if cluster array 1102 represents clusters of genes of expression data array 1002 and if supervising array 1202 is derived from either cluster array 1102 or expression data array 1002 or is constructed to correspond to expression data array 1002 as described more completely below.

It is also possible to compare or correlate dataset 1000, cluster array 1102, and/or supervising array 1202 with a different genetic dataset. To accomplish such comparison or correlation, supervising array 1202 is mapped to a new supervising array corresponding to the experiment metadata of the other genetic dataset in the manner described more completely below.

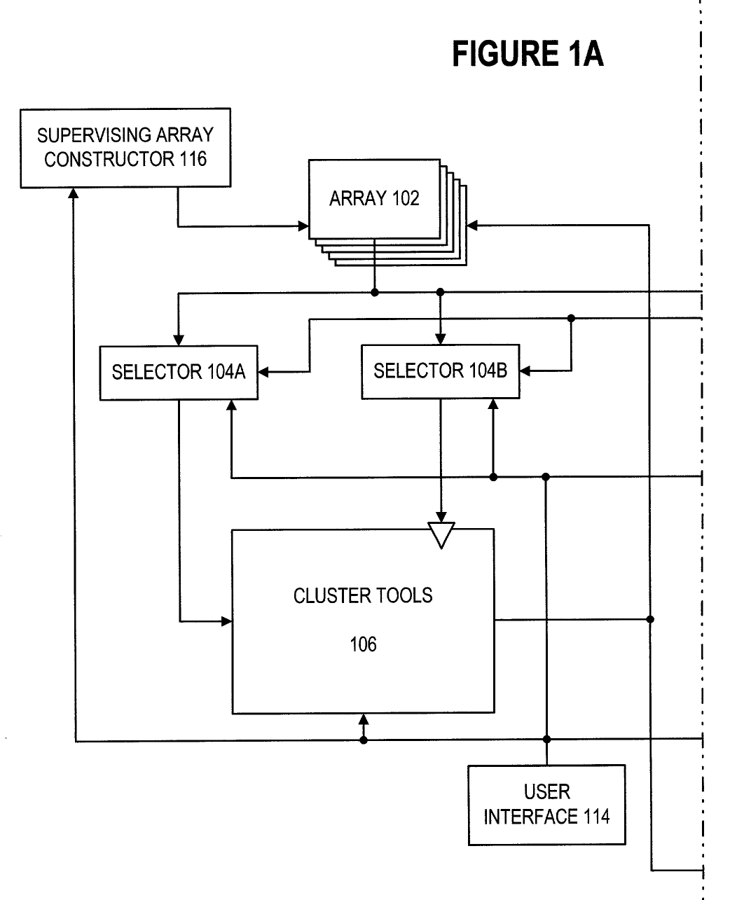

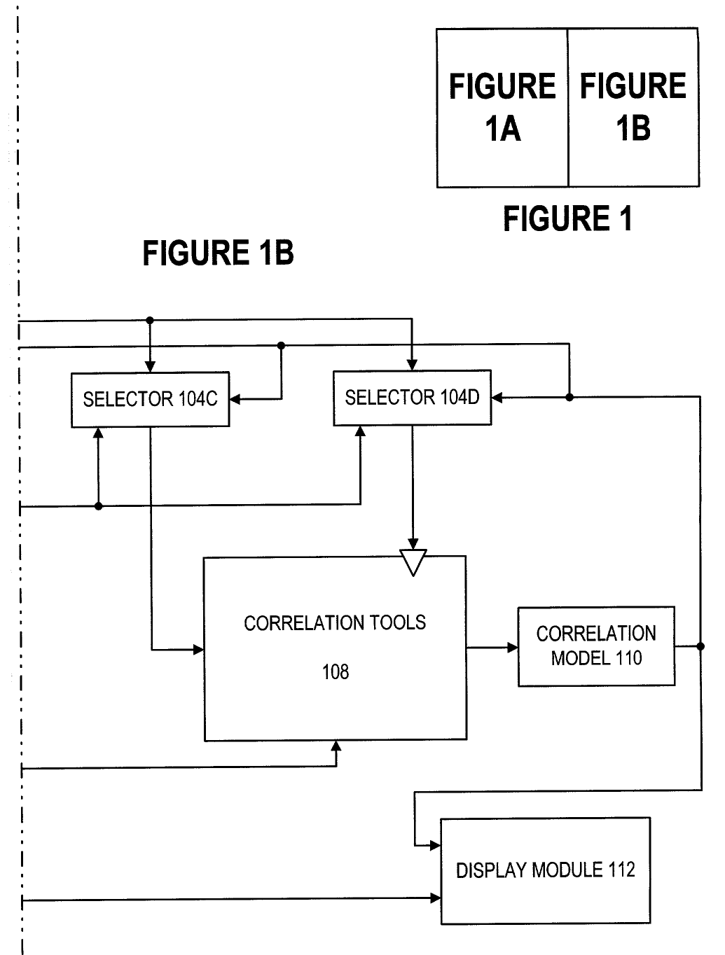

System 100 (FIG. 1) operates on one or more arrays 102, each of which can be an expression data array, a cluster array, or a supervising array. In this illustrative embodiment, expression values in arrays 102 have been normalized, filtered, and imputed in a manner described more completely below. Selectors 104A-D each select one of arrays 102 according to signals provided by a user through a user interface 114. Selector 104A selects one of arrays 102 for processing by cluster tools 106. Selector 104B selects one of arrays 102 as a collection of one or more response variables for use in a manner described below. Cluster tools 106 produce a cluster array such as cluster array 1102 and associated cluster metadata such as cluster metadata 1104. As shown, the resulting cluster array can be displayed on display module 112 and is stored as a new one of arrays 102. Accordingly, the resulting cluster array can be subsequently processed by clustering tools 106 and/or can serve as a collection of response variables selected by selector 104B.

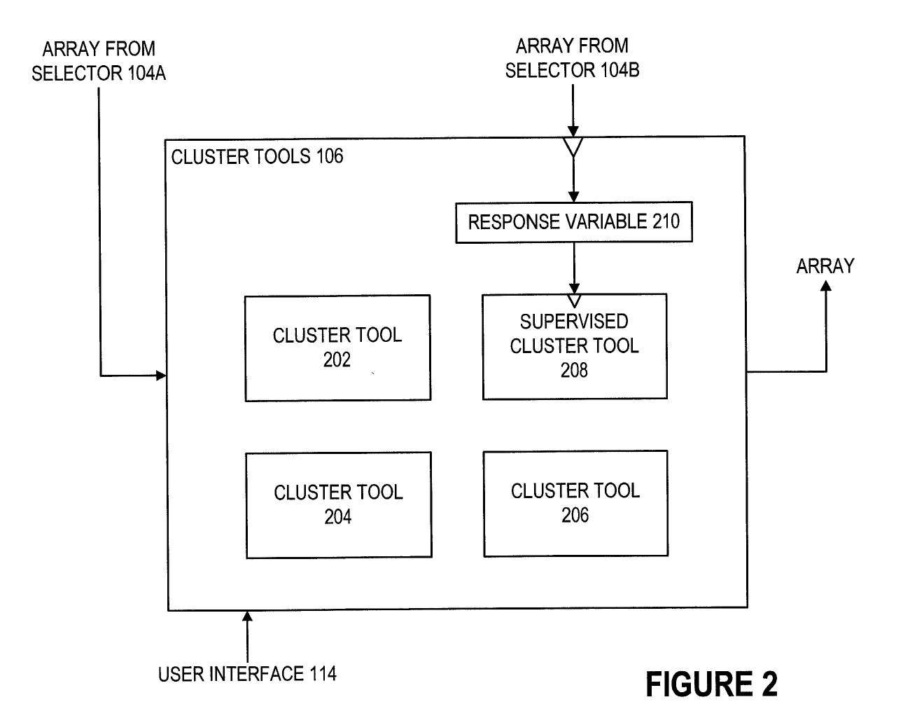

Cluster tools 106 are shown in greater detail in FIG. 2. Cluster tools 106 include cluster tools 202, 204, 206, and 208. Cluster tool 208 is a supervised cluster tool and is described more completely below. Various cluster tools are known and any such cluster tools can be included in cluster tools 106. Additional cluster tools provide greater flexibility and enhance system 100 (FIG. 1). While four (4) cluster tools are shown in cluster tools 106, it is appreciated that fewer or more cluster tools can be included in cluster tools 106. In this illustrative embodiment, cluster tools 106 include the following cluster tools:

The known K-Means cluster tool.

The known K-Mediod cluster tool.

The known Hierarchical Clustering cluster tool.

The known Gene Shaving cluster tool described in Trevor Hastie, Robert Tibshirani, Michael Eisen, Patrick Brown, Doug Ross, Uwe Scherf, John Weinstein, Ash Alizadeh, Louis Staudt, and David Botstein, “Gene Shaving: a New Class of Clustering Methods for Expression Arrays,” available through the World Wide Web at http://www-stat.stanford.edu/˜hastie/Papers/shave.pdf.

The known SOM cluster tool.

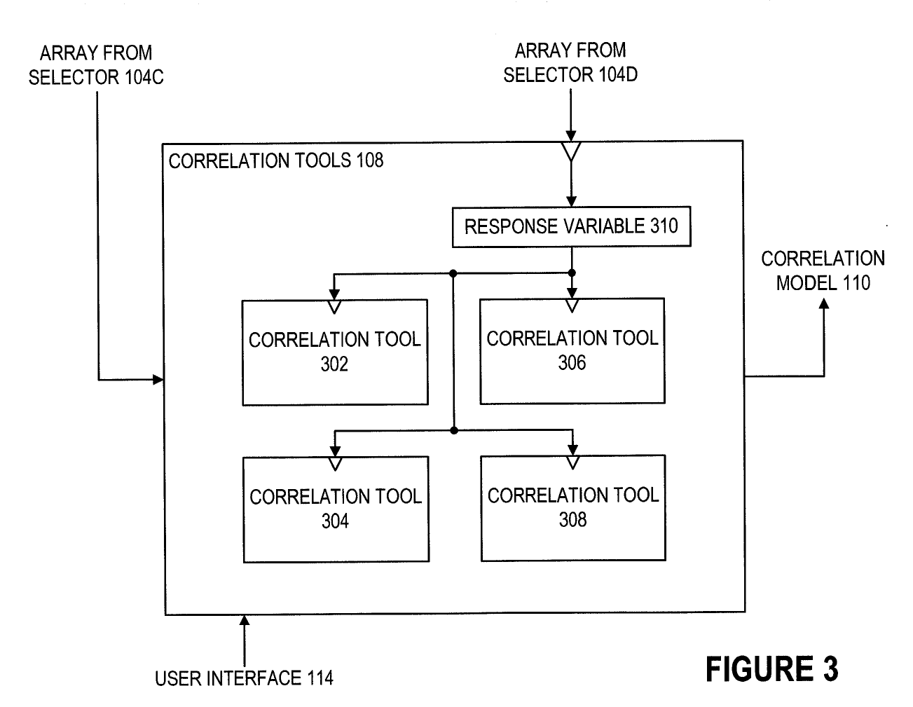

Cluster tool 208 is a supervised cluster tool, such as the known supervised Gene Shaving cluster tool. In particular, supervised cluster tool 208 uses a response variable 210 to guide the formation of clusters from the array received from selector 104A. Supervised cluster tools are known and are only described briefly herein. In general, cluster tools group expression data into clusters of genes or proteins which are similar and/or related to one another. Supervised cluster tools use a response variable as a reference for comparison for determining which gene or proteins are similar and/or related to one another. Supervised cluster tool 208 uses response variable 210 as a reference for comparison of individual rows of the one of arrays 102 selected by selector 104A in generally the manner described below with respect to response variable 310 (FIG. 3). Response variable 210 (FIG. 2) has generally the form of supervising array 1202 (FIG. 12) described above. Accordingly, selector 104B provides arrays in the form of supervising array 1202.

As described above, arrays 102 can include arrays of the types described above with respect to expression data array 1002, cluster array 1102, and supervising array 1202. In other words, selector 104B can select a cluster array such as cluster array 1102 (FIG. 11) whose expression data, either expression data of a member gene of the cluster array or composite expression data such as a weighted average of the member genes, as the response variable. As described above, supervising array 1202 (FIG. 12) can be a one-dimensional array of expression values which is equivalent to a single row of either expression data array 1002 (FIG. 10) or cluster array 1102 (FIG. 11). Accordingly, expression data array 1002 and cluster array 1102 can be thought of as a collection of supervising arrays 1202.

In this illustrative embodiment, selector 104B determines (i) that the selected one or arrays 102 is an array of expression values and (ii) the dimensions of the selected one of arrays 102. If the selected array 102 is an array of expression values, selector 104B provides each row of the selected array as response variable 210 in sequence. The following example is illustrative.

Consider that selector 104A selects an expression data array of the form shown in FIG. 10 as the one of arrays 102 to be processed by cluster tools 106. User interface 114 specifies that supervised cluster tool 208 is to process the selected array, and selector 104B selects a cluster array of the form shown in FIG. 11 for response variable 210. Suppose further that the cluster array selected by selector 104B has ten (10) clusters, i.e., that cluster array 1102 has ten (10) rows, each of which includes composite expression data such as a weighted average of the member genes of each cluster. In this illustrative embodiment, selector 104B provides each of the ten (10) rows of the selected array to cluster tools 106 as response variable 210 in sequence.

For each row of the array selected by selector 104B, supervised cluster tool 208 produces a cluster array of the form described above with respect to FIG. 11 from the array selected by selector 104A. Accordingly, this configuration produces ten (10) cluster arrays.

In an alternative embodiment, user interface 114 allows a user to select one or more rows of such an array selected by selector 104B. In yet another alternative embodiment, the user can extract individual rows of any of arrays 102 and add the individual row as a new array in the form of supervising array 1202 and store the new array in arrays 102. Each such new array can then be selected by selector 104B for use as response variable 210 in the manner described above. Any of these embodiments enable a user to select individual genes or individual gene clusters for use as response variable 210.

Correlation tools 108 determine a degree of correlation between a response variable and genes, in the case of expression data arrays as described with respect to FIG. 10, or between a response variable and gene clusters, in the case of cluster arrays as described with respect to FIG. 11. Selector 104C selects one of arrays 102 for processing by correlation tools 108 and selector 104D selects one of arrays 102 to provide a response variable in the manner described above with respect to selector 104B and response variable 210.

Correlation tools 108 are shown in greater detail in FIG. 3. Correlation tools 108 include correlation tools 302, 304, 306, and 308. Various correlation tools are known and any such correlation tools can be included in correlation tools 108. Additional correlation tools provide greater flexibility and enhance system 100 (FIG. 1). While four (4) correlation tools are shown in correlation tools 108, it is appreciated that fewer or more correlation tools can be included in correlation tools 108. In this illustrative embodiment, correlation tools 108 include the following correlation tools:

The known Tree Harvest correlation tool described in Trevor Hastie, Robert Tibshirani, David Botstein, and Patrick Brown, “Supervised Harvesting of Expression Trees”.

Neural network correlation tools as described in Robert Tibshirani, “A comparison of some error estimates for neural network models” available through the World Wide Web at http://www-stat.stanford.edu/˜tibs/ftp/harvest.pdf.

The known SVM (Support Vector Machine) correlation tool described in Michael P. S.

Brown, William Noble Grundy, David Lin, Nello Cristianini, Charles Walsh Sugnet, Terrence S. Furey, Manuel Ares, Jr., and David Haussler, “Knowledge-based analysis of microarray gene expression data by using support vector machines,” Proceedings of the National Academy of Sciences, vol. 97, no. 1, pp. 262-67 (Jan. 4, 2000).

The known SAM (Significance Analysis of Microarrays) cluster tool described in V. Tusher, R. Tibshirani, and C. Chu, “Significance analysis of microarrays applied to ionizing radiation response,” Proceedings of the National Academy of Sciences, 2001. First published Apr. 17, 2001, 10.1073/pnas.091062498.

In addition, correlation tools 108 include a response variable 310 as a reference for determination of respective degrees of correlation. Each of the correlation tools determines a degree of correlation between each row of the one of arrays 102 selected by selector 104C and response variable 310. The degree of correlation is determined according to the particular configuration of the correlation tool. As described above with respect to response variable 210 (FIG. 2), response variable 310 (FIG. 3) is of the form described above with respect to supervising array 1202 (FIG. 12).

As described above, supervising array 1202 (FIG. 12) can include expression value data, class label data, survival time data, or time series data. It is appreciated that other types of data can be used as response variables for both supervised cluster tools and correlations tools. These four (4) types of response variables are merely selected as illustrative examples. Each supervised cluster tool of cluster tools 106 and each correlation tool 108 expects a response variable of a certain format. Accordingly, user interface 114 ensures that the one of arrays 102 selected as a response variable is of the type expected by the corresponding selected supervised cluster tool or correlation tool.

If the selected correlation tool expects, and selector 104D selects, a response variable 310 which is a collection of expression value data, expression values of each of the columns of response variable 310 are compared to, or mathematically combined with, a corresponding one of the columns of a row of the selected array. In one simple illustrative example, a correlation score for a particular row of genetic data is the sum of squared differences between individual gene expression values in the row and corresponding expression values in response variable 310. The row with the lowest sum of squared differences is the row with the highest correlation. The degree of correlation can be represented as a score corresponding to the particular row of the selected expression data.

In other correlation tools, a correlation model is formed from the expression data array selected by selector 104C. Such a correlation model represents mathematical relationships between various rows of the selected expression data array to predict response variable 310. For example, if expression data array 1002 contains genetic expression data and supervising array 1202 contains data corresponding to a human condition indicated in experiment metadata 1004, a correlation model for expression data array 1002 and supervising array 1202 specifies relationships between one or more genes of expression data array 1002 which reasonably accurately predict the values stored in supervising array 1202. For example, if supervising array 1202 represents survival time, the resulting correlation model specifies a mathematical formula for predicting a relative risk of mortality for a particular patient based on the patient's genetic expression data. Such relative risk of mortality can be represented as a curve representing time vs. likelihood of survival for various amounts of time. From such a curve, life expectancy of the patient can be estimated.

Of course, other measurements of correlation are known and can be used.

If the selected correlation tool expects, and selector 104D selects, a response variable 310 which is a collection of class labels, the selected correlation tool determines a degree of correspondence among expression values for experiments belonging to each of the classes. For example, if most instances of a particular gene have high expression values for experiments of a particular class representing a particular condition, it can be likely that the gene influences the particular condition.

If the selected correlation tool expects, and selector 104D selects, a response variable 310 which is a collection of survival times, the selected correlation tool correlates survival times to respective expression data at each row in generally the manner described above with respect to expression value response variables. However, in some correlation tools, indication that survival of a particular patient beyond a given survival time is uncertain can be used to attribute appropriate significance to the given survival time in modeling a survival time curve.

If the selected correlation tool expects, and selector 104D selects, a response variable 310 which is a collection of time series data, the selected correlation tool correlates the measured condition with each row of the selected one of arrays 102 over time. In particular, the selected correlation tool determines a measure value for each time for which expression data is available, either as directly specified in response variable 310 or interpolated from values specified in response variable 310. Once a measured value is determined for each time for which expression data exists, the selected correlation tool correlates the measured values to respective expression data at each row in generally the manner described above with respect to expression value response variables.

The results of correlation by the selected correlation tool are stored in a correlation model 110 (FIG. 1). Correlation model 110 specifies a relationship between one or more rows of the array selected by selector 104C and response variable 310 (FIG. 3). Typically, correlation model 110 specifies a mathematical model by which individual values of response variable 110 can be predicted using corresponding expression data of one or more rows of the selected array. Alternatively, correlation model 110 (FIG. 1) can specify, for each row in the one of arrays 102 selected by selector 104C, a score which represents a degree of correlation with response variable 310 as selected by selector 104D. Such scores can be used as a mathematic model for predicting response variable as each score can be used as a respective row weight to form a weighted average, for example.

Correlation model 110 can be displayed in display module 112 for analysis by the user. In addition, correlation model 110 can be used by selectors 104A-D to further analyze rows of high correlation in a manner described more completely below.

The following is an illustrative example of cross-dataset analysis using correlation model 110. Consider that response variable 310 represents survival times for patients with a particular ailment, e.g., prostate cancer. Consider further that correlation model 110 accurately predicts relative risk of dying at various times for any individual with expression data given from a particular one of arrays 102. If another one of arrays 102 pertains to an entirely different dataset of different experiments for which no survival data is available, such survival times can be inferred. Correlation model 110 can be used to create an array of hypothetical survival data corresponding to the second one of arrays 102 for subsequent analysis, e.g., to perform supervised clustering to determine whether perhaps other genes correlate to those involved in correlation model 110 from the first of arrays 102.

Thus, arrays 102 can include expression data arrays, cluster arrays, and supervising arrays and can include arrays resulting from processing by cluster tools 106 and can select arrays according to degrees of correlation.

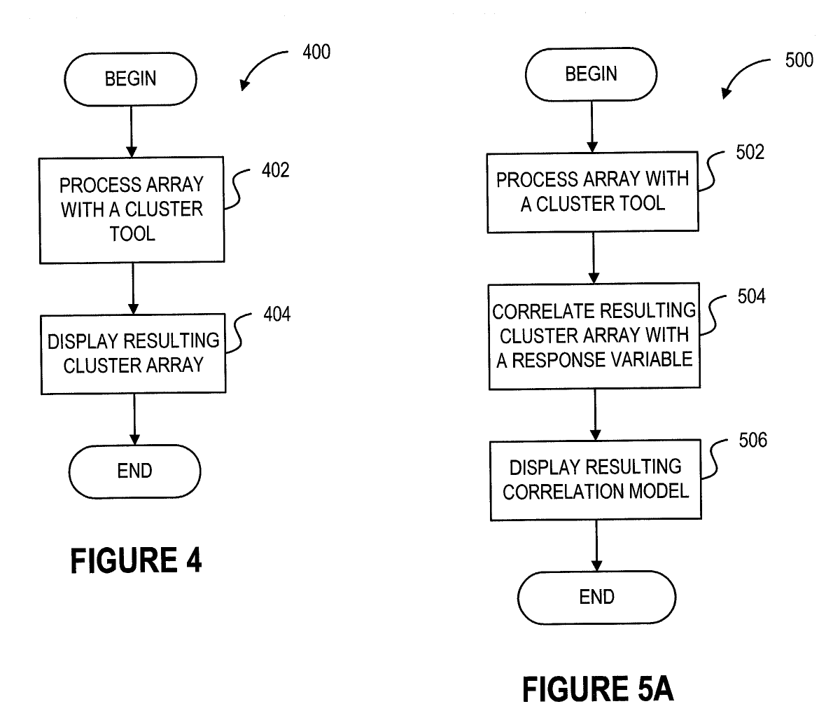

A particularly simple application of system 100 is shown as logic flow diagram 400 (FIG. 4). In step 402, selector 104A selects one of arrays 102 for processing according to one of cluster tools 202-208 (FIG. 2) to produce a cluster array. In step 402 (FIG. 4), display module 112 displays the resulting cluster array to the user.

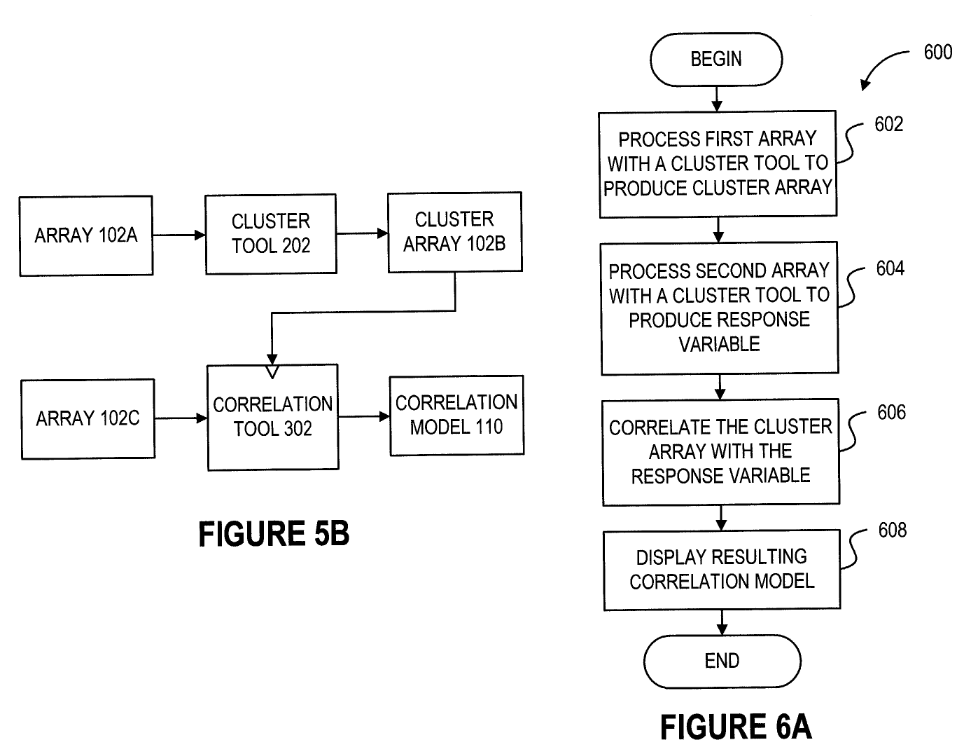

Logic flow diagram 500 (FIG. 5A) shows processing of an expression data array in which the results of one processing step is further analyzed with an additional processing step. Processing according to logic flow diagram 500 is summarized in FIG. 5B. In particular, system 100 processes a selected expression data array 102 (e.g., expression data array 102A) by a selected cluster tool (e.g., cluster tool 202) to produce a cluster array 102B in step 502 (FIG. 5A). Cluster array 102B is stored in arrays 102.

In step 504 (FIG. 5A), cluster array 102B is correlated with a response variable 102C. In particular, selector 104C selects cluster array 102B from arrays 102, and selector 104D selects response variable 102C from arrays 102. The result is stored in correlation model 110 and is displayed in display module 112 for the user in step 506 (FIG. 5A). The advantage of processing expression data arrays according to logic flow diagram 500 is significant. It appears that many human conditions are effected not by any one gene in isolation but rather by a number of genes. A single correlation tool applied to genetic data corresponding to all such genes may not accurately indicate the interplay between the various genes affecting the condition. However, by using a cluster tool, various clusters of the genes can be gathered using one measure of interrelation between genes and correlation to the response variable of each of the various clusters can be measured using a separate standard of correlation. The result—as shown in FIG. 5B—is a powerful tool for correlating genetic expression data to conditions affected by clusters of multiple genes.

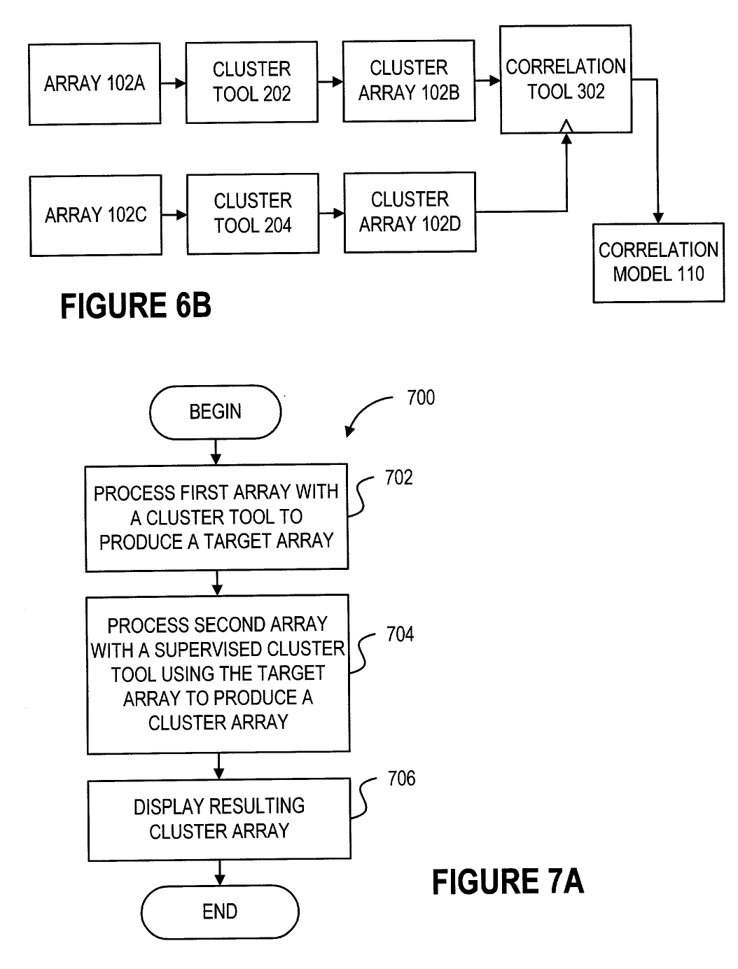

Logic flow diagram 600 (FIG. 6A) shows use of a clustering tool to create response variables for subsequent processing. Processing according to logic flow diagram 600 is summarized in FIG. 6B. In step 602, system 100 processes a first one of arrays 102 (e.g., array 102A in FIG. 6B) using a cluster tool (e.g., cluster tool 202) to produce a cluster array 102B in the manner described above with respect to steps 402 and 502. Cluster array 102B is stored in arrays 102 for subsequent processing.

In step 604 (FIG. 6A), system 100 processes a second one of arrays 102, e.g., array 102C, using another cluster tool, e.g., cluster tool 204, to produce a second cluster array 102D.

In step 606 (FIG. 6A), system 100 processes cluster array 102B using a correlation tool, e.g., by selecting cluster array 102B using selector 104C and applying cluster array 102C to correlation tool 302. In step 606, response variable 310 is selected from clusters of cluster array 102D. For example, each of the clusters of cluster array 102D is used as response variable 310 in a respective iterative performance of step 606. Alternatively, the user can select individual clusters of cluster array 102D for use as response variables in respective iterative performances of step 606.

In step 608, system 100 displays each of the one or more resulting correlation models 110 to the user in display module 112. Thus, according to logic flow diagram 600 (FIG. 6A), the user can compare clusters of an expression data array, e.g., array 102A (FIG. 6B), with clusters of another expression data array, e.g., array 102C. In particular, by selecting a cluster from cluster array 102D as the response variable for correlation tool 302, correlation model 110 presents a degree of correlation between the selected cluster of cluster array 102D and clusters of cluster array 102B. In effect, a cross-correlation between cluster arrays 102B and 102D is determined.

Such cross-correlation can be particularly useful in comparing expression data from different datasets. Due to the expense of obtaining expression data, some datasets can include relatively few experiments and thus providing results of marginal reliability. The ability to combine analysis of expression data from multiple datasets allows existing datasets to be analyzed in conjunction with new datasets to provide significantly more reliable results with only incremental costs associated with new datasets.

Cross-correlation in the manner shown in FIGS. 6A-B provides an indication regarding whether clusters of array 102A are also significant within array 102C. Uses of such cross-correlation include (i) comparing data pertaining to similar studies but collected with different methodologies; (ii) comparing data pertaining to similar studies but conducted by different laboratories or from subjects of different demographics; and (iii) comparing data pertaining to similar, but different, studies—e.g., studies regarding different types of cancer.

While it is shown that cluster tool 202 processes array 102A and cluster tool 204 processes array 102C, it is appreciated that the same cluster tool can be used or that the same array can be processed. For example, the same cluster tool, e.g., cluster tool 202, can process both array 102A and 102C. Similarly, cluster tools 202 and 204, can process the same array, e.g., array 102A, to produce cluster arrays 102C and 102D. Applying different cluster tools to the same dataset enables comparison of the cluster tools themselves.

The flexibility of system 100 as illustrated in FIGS. 6A-B is significant. Expression data arrays and datasets vary significantly as does the manner in which various genes affect various conditions. No one cluster tool is best for all datasets. Similarly, no one correlation tool is best for all datasets. However, use of results of one cluster or correlation tool for analysis in another cluster or correlation tool enables the user to empirically determine the significance of various genes represented in various datasets.

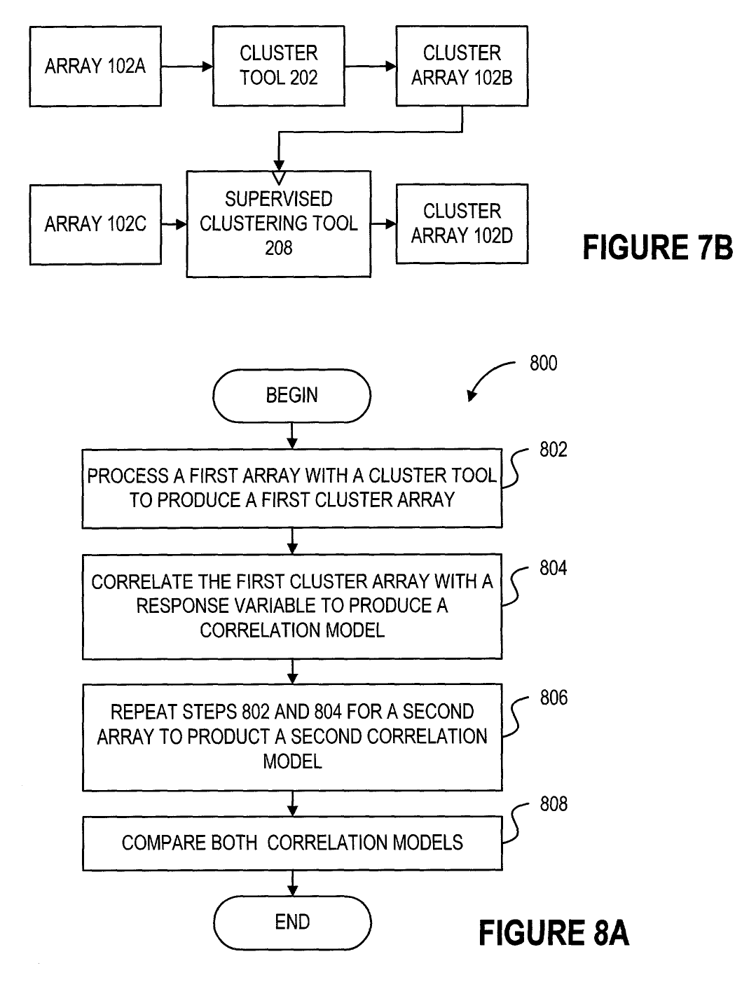

Logic flow diagram 700 (FIG. 7A) shows another multi-stage analysis of genetic data according to the present invention. Processing according to logic flow diagram 700 is summarized in FIG. 7B.

In step 702, system 100 processes a first one of arrays 102 (e.g., array 102A in FIG. 7B) using a cluster tool (e.g., cluster tool 202) to produce a cluster array 102B in the manner described above with respect to steps 402, 502, and 602. Cluster array 102B is stored in arrays 102 for subsequent processing.

In step 704 (FIG. 7A), system 100 processes a second one of arrays 102, e.g., array 102C, using a supervised cluster tool, e.g., supervised cluster tool 208, using one or more clusters of cluster array 102B as response variable 210 (FIG. 2) to produce additional cluster arrays such as cluster array 102D (FIG. 7B). In step 704 (FIG. 7A), response variable 210 is selected from clusters of cluster array 102B. For example, each of the clusters of cluster array 102B is used as response variable 210 in a respective iterative performance of step 704. Alternatively, the user can select individual clusters of cluster array 102B for use as response variables in respective iterative performances of step 704.

In step 706 (FIG. 7A), system 100 displays the one or more resulting cluster arrays in display module 112 for viewing by the user. Thus, according to FIGS. 7A-B, clusters of one array are used as response variables of a supervised cluster tool for processing another array. If the user has determined that a particular cluster of cluster array 102B is significant, e.g., correlates strongly with a particular human condition, the user can use that cluster in the manner shown in FIGS. 7A-B to identify similar patterns in the second array, e.g., array 102C. In addition, through supervised cluster tool 208, the user can determine whether a cluster of cluster array 102C, which is believed to be significant in array 102A, is also significant in array 102C.

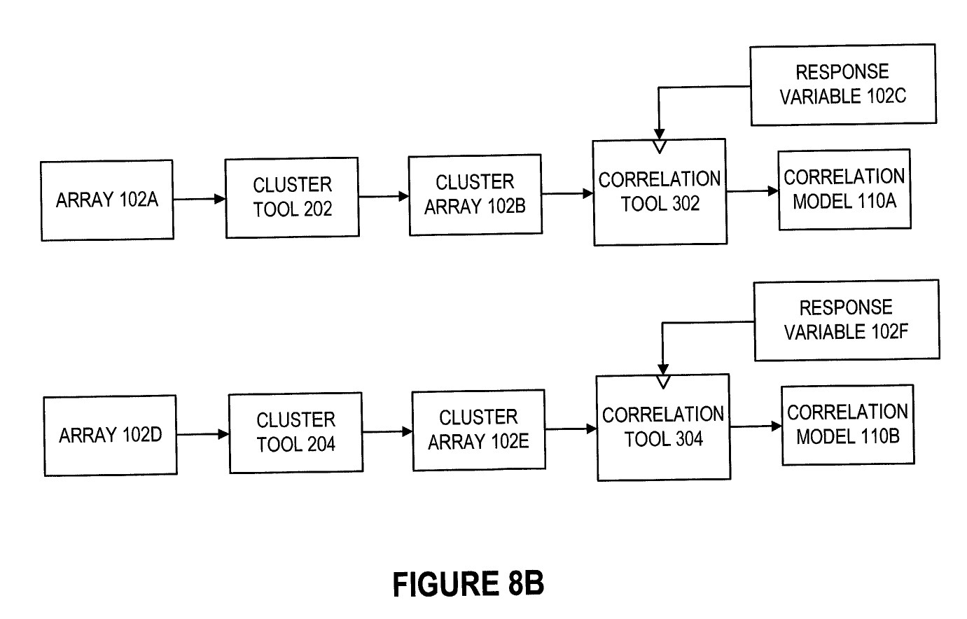

Logic flow diagram 800 (FIG. 8A) shows a multi-step process for analysis of genetic data in accordance with the present invention. Logic flow diagram 800 is summarized in FIG. 8B.

In step 802, system 100 processes a first one of arrays 102, e.g., array 102A, according to a selected one of cluster tools 106, e.g., cluster tool 202, to produce a cluster array 102B in generally the manner described above with respect to steps 402, 502, 602, and 702.

In step 804, system 100 processes cluster array 102B with a correlation tool, e.g., correlation tool 302, using a response variable 102C to produce a correlation model 110A. Thus, correlation model 110A represents various degrees of correlation between respective clusters of cluster array 102B and response variable 102C.

In step 806, system 100 repeats steps 802-804 for a second one of arrays 102, e.g., array 102D. In particular, system 100 processes array 102D according to a selected one of cluster tools 106, e.g., cluster tool 204, to produce a second cluster array 102E in generally the manner described above with respect to steps 402, 502, 602, and 702. In addition, system 100 processes cluster array 102E with a correlation tool, e.g., correlation tool 304, using a response variable 102F to produce a second correlation model 110B. Thus, correlation model 110B represents various degrees of correlation between respective clusters of cluster array 102E and response variable 102F.

In step 808, the user compares correlation models 110A-B. Comparison can be visual by viewing displays of correlation models 110A-B in display module 112 or can be cross-correlation of the correlation scores represented in correlation model 11 OA-B, for example. By selecting arrays 102A and 102D which are related and selecting response variables 102C and 102F accordingly, the user can determine if genes are significant across different conditions. For example, array 102A and response variable 102C can be selected to determine genes which are significant for breast cancer and array 102D and response variable 102F can be selected to determine genes which are significant for ovarian cancer. In this illustrative example, comparison of correlation models 110A-B determines whether the same genes or same clusters are significant in both breast and ovarian cancers.

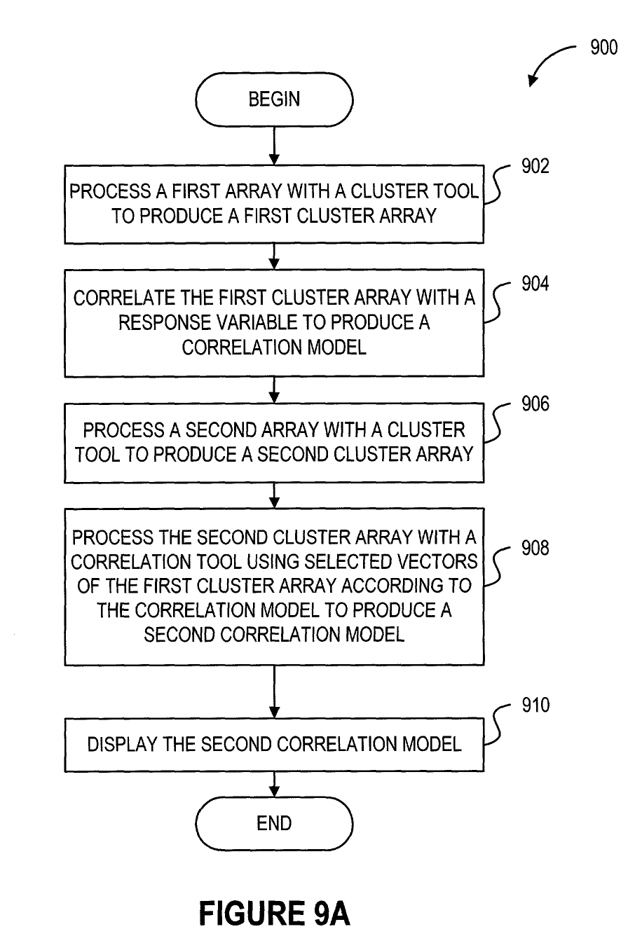

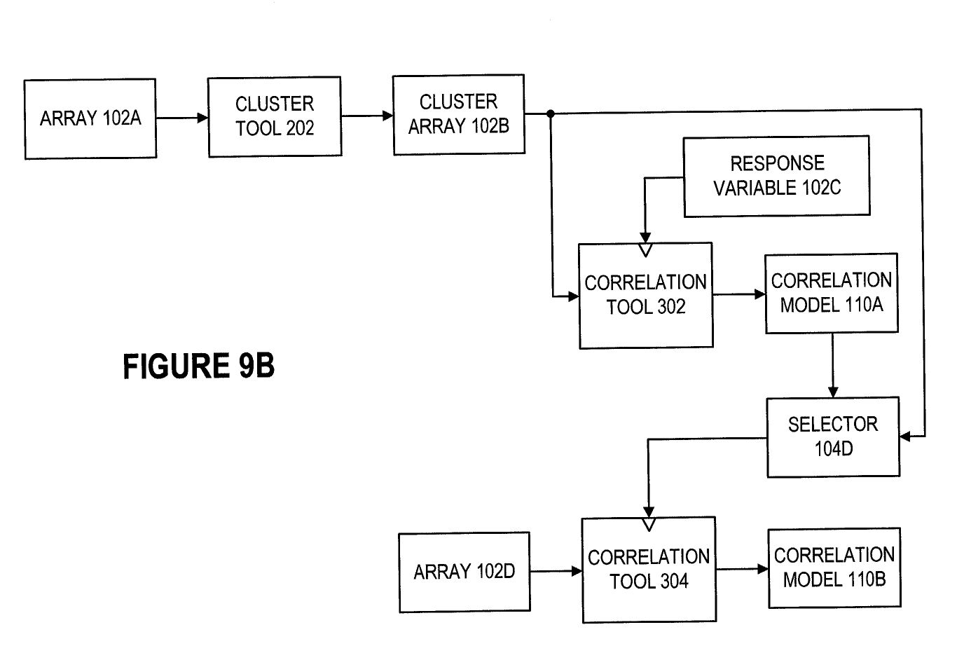

Logic flow diagram 900 (FIG. 9A) shows a multi-step process for analysis of genetic data in accordance with the present invention. Logic flow diagram 900 is summarized in FIG. 9B.

In step 902, system 100 processes a first one of arrays 102, e.g., array 102A, according to a selected one of cluster tools 106, e.g., cluster tool 202, to produce a cluster array 102B in generally the manner described above with respect to steps 402, 502, 602, 702, and 802.

In step 904, system 100 processes cluster array 102B with a correlation tool, e.g., correlation tool 302, using a response variable 102C to produce a first correlation model 110A. Thus, correlation model 110A represents various degrees of correlation between respective clusters of cluster array 102B and response variable 102C.

In step 906, system 100 processes a second array 102D using a correlation tool, e.g., correlation tool 302, to produce a second correlation model 110B. The response variable of correlation tool 302 is selected by selector 104D from cluster array 102B according to degrees of correlation represented in correlation model 110A. In one embodiment, only one response variable is selected from cluster array 102B, namely, the cluster of cluster array 102B corresponding to the highest degree of correlation as represented in correlation model 110A. In other embodiments, multiple clusters of cluster array 102B are selected by selector 104D as respective response variables of correlation tool 302 to produce respective correlation models.

In step 908, system 100 displays correlation model 110B to the user through display module 112. Thus, according to FIGS. 9A-B, clusters of array 102A which have a strong correlation to response variable 102C are selected as response variables for analyzing array 102D. Such enables correlation between arrays 102A and 102D to be determined. Determining such correlation is particularly useful in correlating datasets derived from different gene chips or from different laboratories and in correlating new datasets with older, extensively studied datasets.

Display Cross Referencing

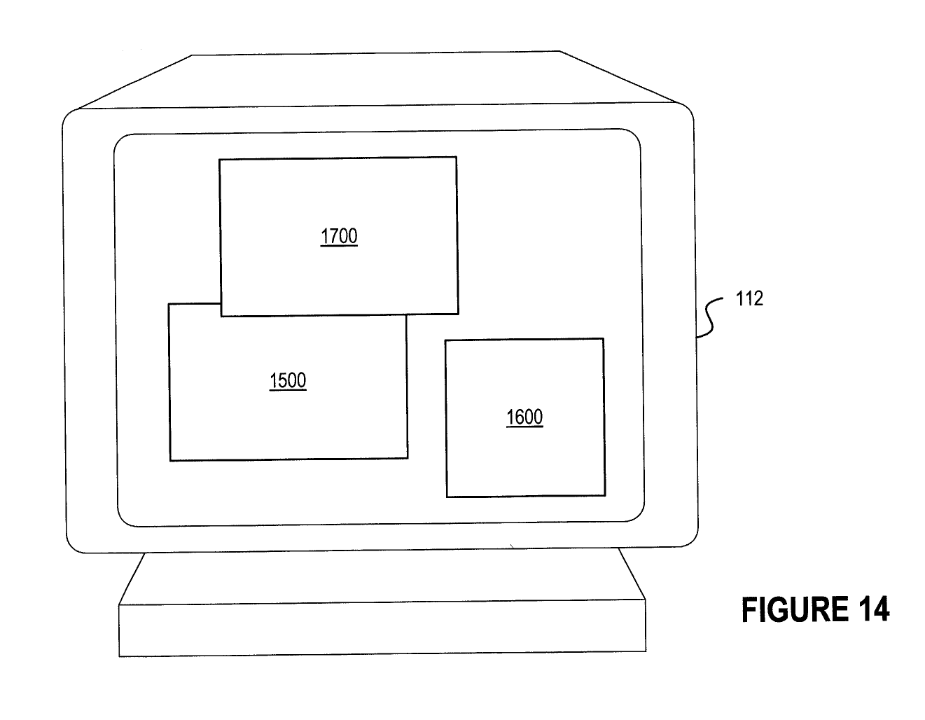

As described above, display module 112 (FIG. 1) shows one or more displays of expression data, representing various results of analysis of such expression data in the manner described above. Display module 112 is shown in greater detail in FIG. 14. Display module 112 can be generally any computer display including, for example, a cathode-ray tube (CRT) or a liquid crystal display (LCD) with accompanying control circuitry. For illustration purposes, display module 112 is shown to include three (3) displays as overlapping windows. In particular, displays 1500, 1600, and 1700 are shown.

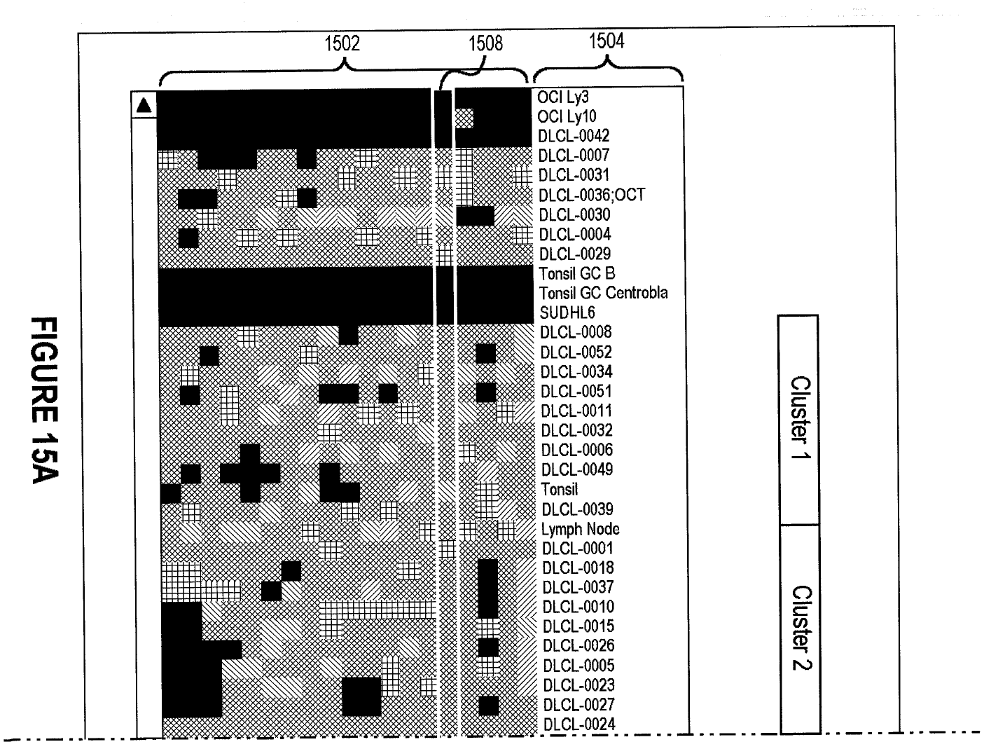

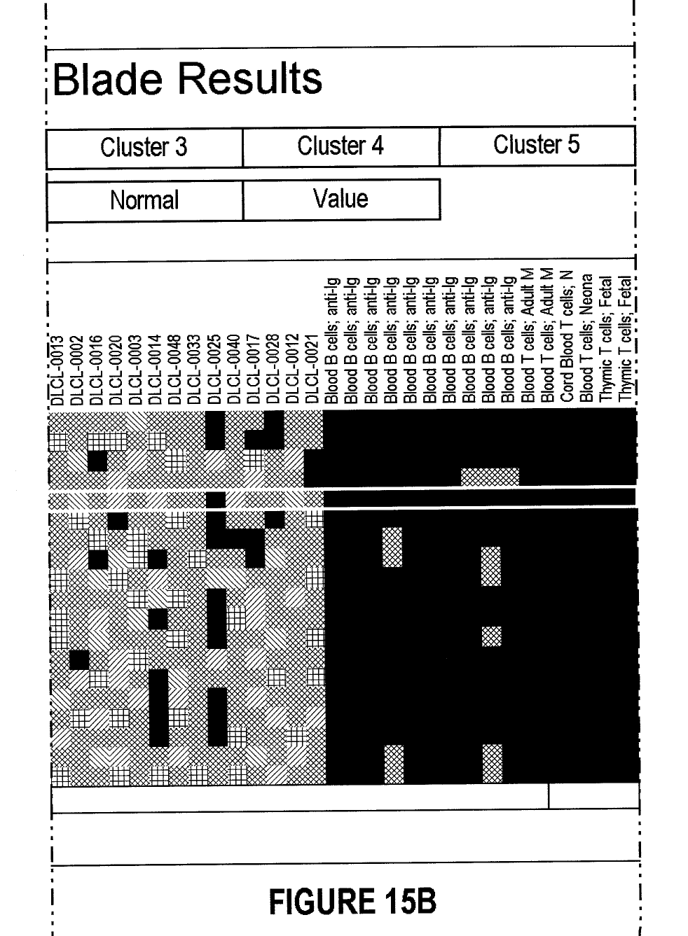

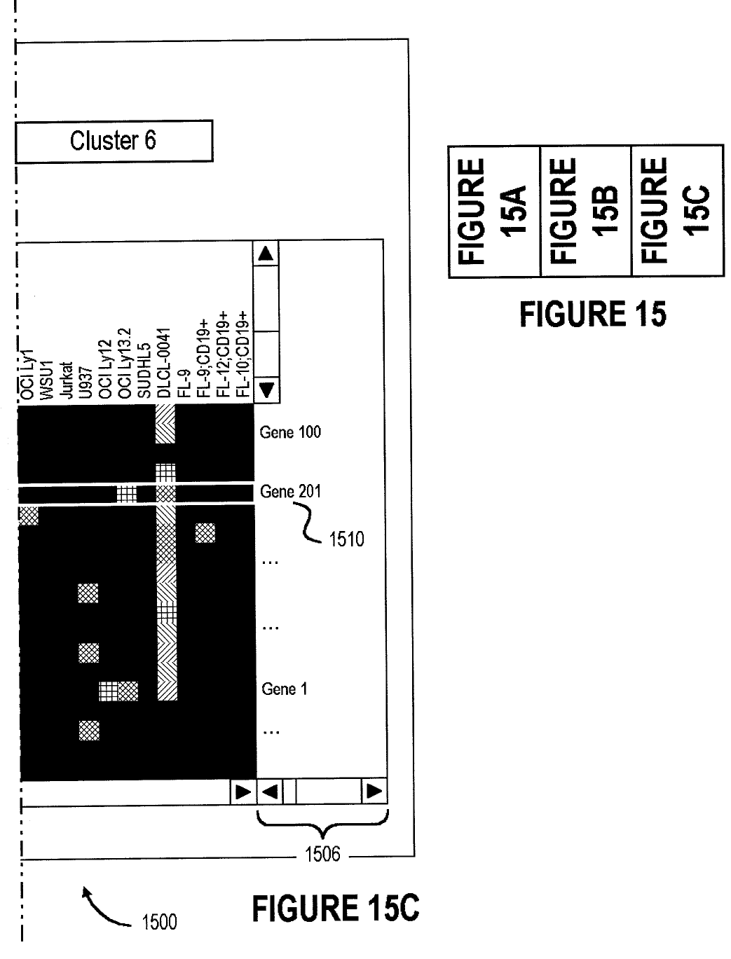

Display 1500 (FIG. 15) displays the results of processing by cluster tool 106. Expression data 1502 represents each expression value, or alternative each of a number of ranges of expression values, as a respective color. Experiment labels 1504 include brief descriptions of respective experiments extracted from experiment metadata 1004 (FIG. 10). Expression labels 1506 (FIG. 15) include brief descriptions of respective clusters of expression data 1502 extracted from expression metadata 1006 (FIG. 10).

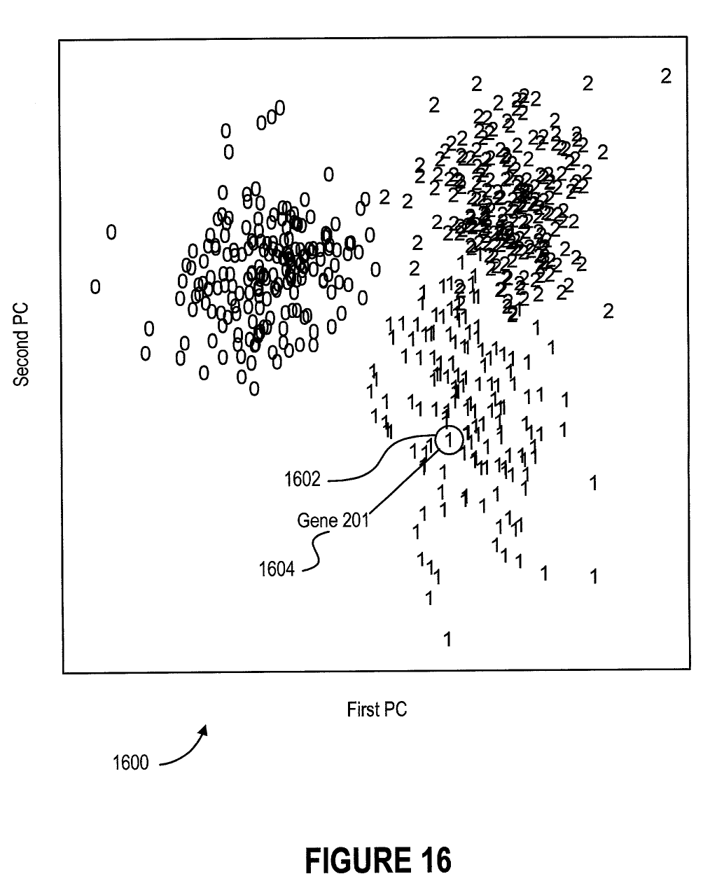

Display 1600 (FIG. 14) is shown in greater detail in FIG. 16. Display 1600 represents a linear discriminant analysis (LDA) of expression data. Each numeral represents a member gene of one of three clusters. Each of the clusters is identified by a numeral identifier, e.g., 0, 1, or 2. The specific position of each numeral within display 1600 is determined according to the expression data of the member gene of the cluster corresponding to the numeral. The position is determined using LDA which is known and conventional and is not described further herein.

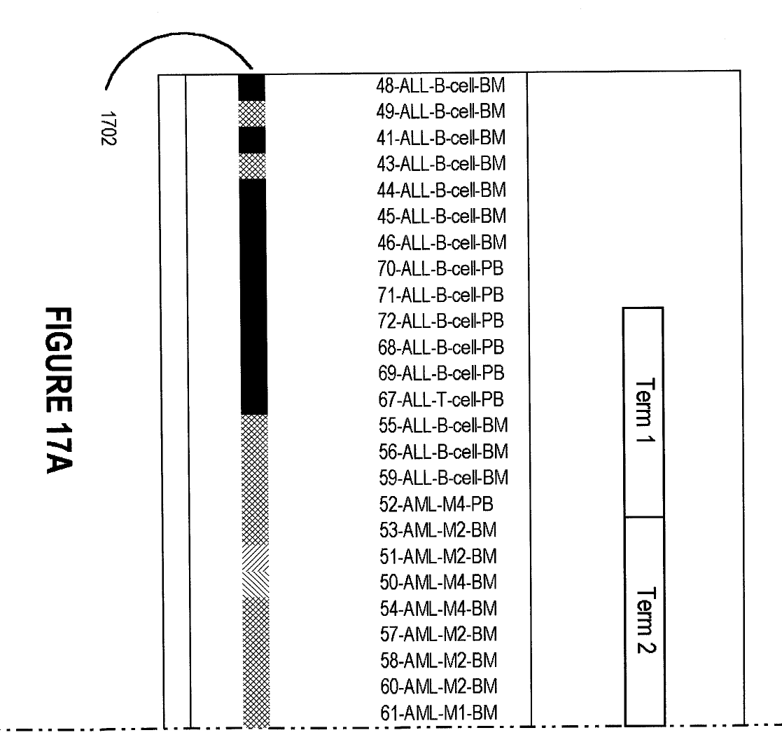

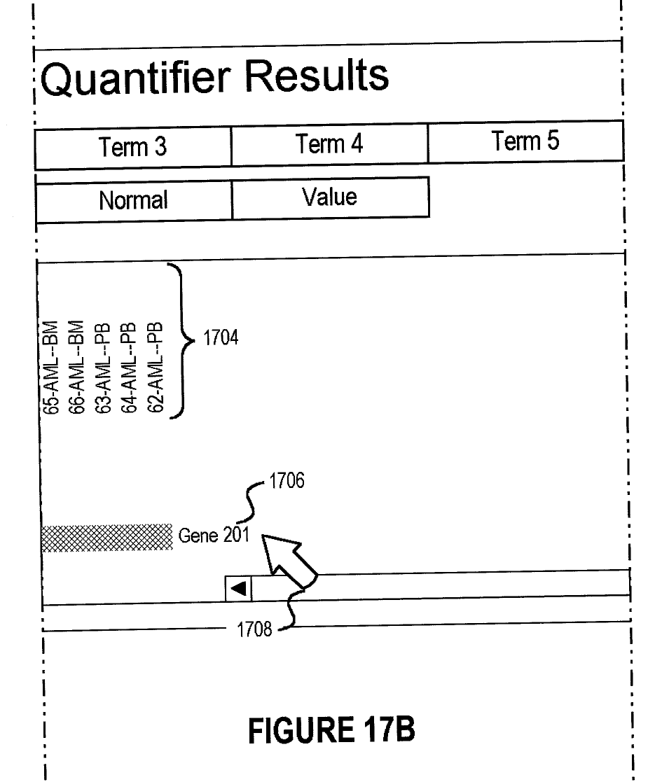

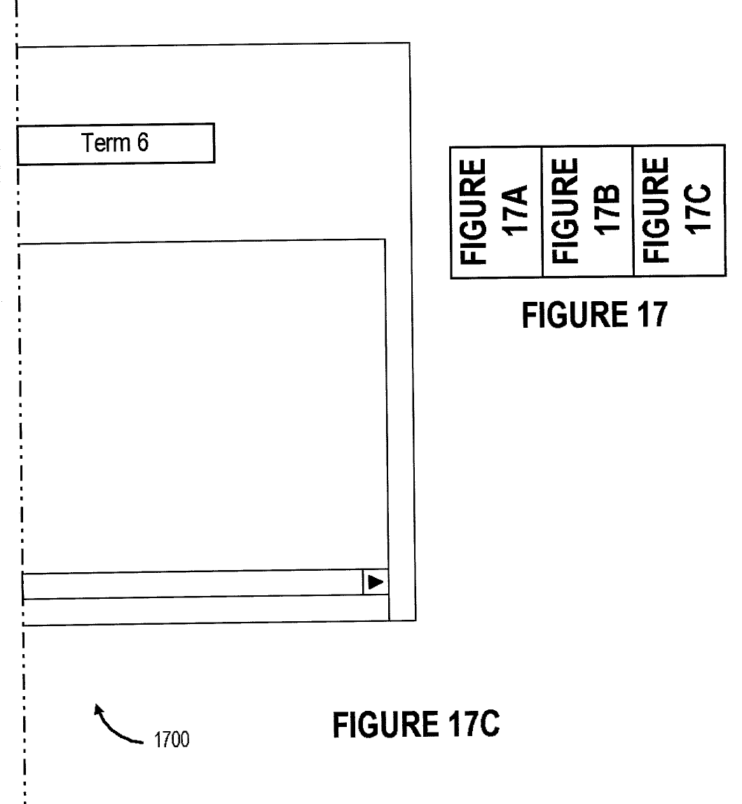

Display 1700 (FIG. 14) is shown in greater detail in FIG. 17. Display 1700 represents displayed results of correlation tool 108 (FIG. 1). A color bar 1702 shows expression data for a particular row of expression data array 1002 (FIG. 10) and can alternatively represent correlation scores of the expression data. Experiment labels 1704 (FIG. 17) are brief descriptions of experiments extracted and/or derived from experiment metadata 1004 (FIG. 10). Expression label 1706 (FIG. 17) is a brief description of the row of expression data array 1002 (FIG. 10) shown in display 1700 (FIG. 17) and is extracted and/or derived from expression metadata 1006 (FIG. 10).

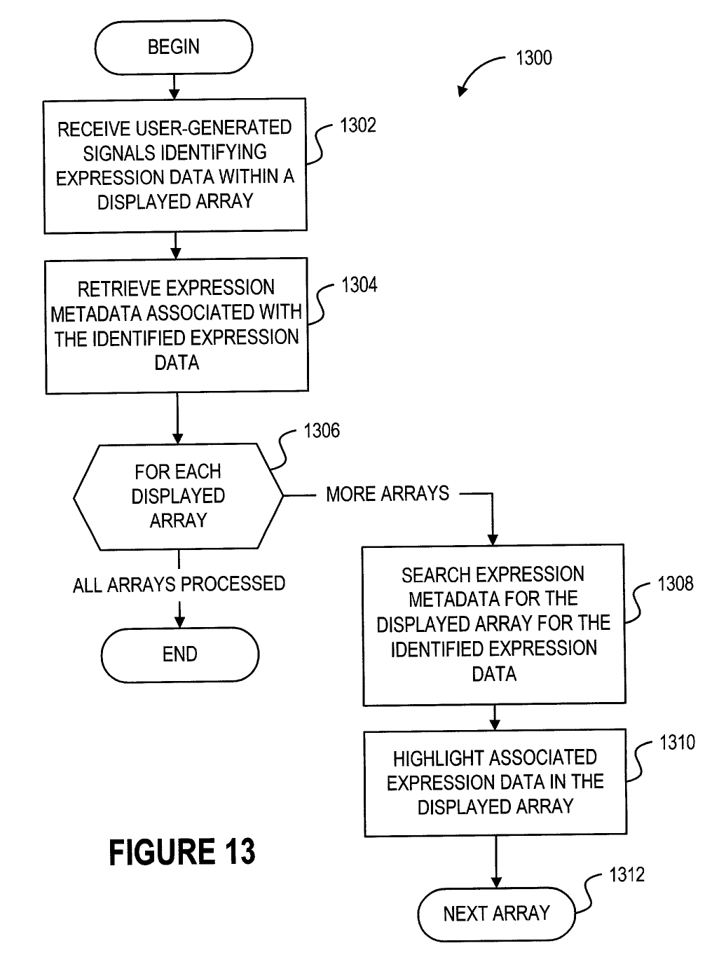

To facilitate interpretation of the multiple, simultaneous displays in display module 112 (FIG. 14), display module 112 and user interface 114 cooperate to provide an interactive display correlation user interface which is illustrated by logic flow diagram 1300 (FIG. 13). In particular, user interface 114 includes one or more user-operated data input devices such as an electronic mouse, trackball, touch-sensitive screen, tablet, voice or speech recognition circuitry and logic, or generally any user input device. By physical manipulation of such a user input device, the user generates and communicates signals to user interface 114.

In step 1302 (FIG. 13), user interface 114 (FIG. 1) receives user generated signals identifying a row of expression data in one of the displays of display module 112. In this illustrative example, the user positions a cursor 1708 (FIG. 17) within display 1700 over expression label 1706 and presses a button or otherwise actuates a user input device in a conventional manner to identify expression label 1706. Accordingly, user interface 114 identifies the specific row of expression data identified by expression label 1706 as the expression row of interest. In this illustrative example, the expression row of interest is a gene whose name is “Gene 201.” User interface 114 makes such a determination in step 1304 (FIG. 13) by reference to expression metadata 1006 if the displayed expression data in display 1700 is of the form described above with respect to FIG. 10 or by reference to cluster metadata 1104 if the display expression data in display 1700 is of the form described above with respect to FIG. 11.

Loop step 1306 and next step 1312 define a loop in which user interface 114 process each display of display module 112 according to steps 1308-1310. During each iteration of the loop of steps 1306-1312, the particular display processed by user interface 114 is sometimes referred to as the subject display.

In step 1308, user interface 114 locates the expression row of the subject display which corresponds to the expression row identified by the user. In step 1310, user interface 114 highlights the expression row located in step 1308. In the illustrative example shown in FIGS. 14-17, the loop of steps 1306-1312 has the following effect.

In this illustrative example, the user identified an expression row corresponding to Gene 201 as shown in FIG. 17. In processing display 1500 (FIG. 15), user interface 114 locates expression row 1510 by reference to associated expression labels 1506 or, alternatively, by reference to the expression or cluster metadata on which expression labels 1506 are based. In step 1310 for display 1500, user interface 114 causes display module 112 to highlight expression row 1510, e.g., by displaying a rectangle 1508 which encloses expression row 1510. Of course, user interface 114 and display module 112 can highlight expression row 1510 in other ways. For example, display module 112 can (i) brighten expression row 1510, e.g., by modifying intensity and/or saturation of the display of expression row 1510 in HSI (hue saturation intensity) colorspace; (ii) cause expression row 1510 to blink momentarily; (iii) redraw expression row 1510 with larger colored elements, e.g., with a height 50% larger than other expression rows; and/or (iv) draw one or more arrows pointing at expression row 1510.

In processing display 1600 (FIG. 16), user interface 114 locates the numeral representing the selected expression row. In this illustrative embodiment, the selected expression row is represented in display 1600 by a numeral “1”, e.g., numeral 1602. To highlight numeral 1602, user interface 114 causes display module 112 to draw a circle around numeral 1602 as shown and connects the circle to a label 1604 which identifies the selected expression row. Of course, user interface 114 can highlight numeral 1602 in other manners. For example, user interface 114 can (i) redraw numeral 1602 in a color different than others of the same numeral face value; (ii) cause numeral 1602 to blink; (iii) redraw numeral 1602 in a different font, a different font weight, and/or a different font size; (iv) enclose numeral 1602 with a different shape; and/or (v) draw one or more arrows pointing at numeral 1602.

After the loop of steps 1308-1312 completes processing of all displays in display module 112, processing according to logic flow diagram 1300 completes.

Interactive highlighting across displays in the manner described above is particularly helpful for viewing results of system 100. In particular, a single expression array can be processed by different cluster tools and the user can quickly and easily determine by juxtaposition of the resulting cluster arrays in display module 112 and clicking on various clusters to determine whether the results of the various cluster tools were comparable. In short, processing in the manner described above with respect to logic flow diagram 1300 provides a quick, easy, and intuitive solution to providing answers to questions of the user such as “What is this?” and “Where is this in the other display?”

Filtering and Imputation

To maximize accuracy of clustering and correlation processing in the manner described above, it is preferred that arrays 102 are preprocessed to ensure that missing data is either (i) excluded or (ii) imputed prior to such processing. In general, genetic and proteomic expression data include two components: a measure of a degree of expression of a particular element and a measure of reliability of the degree of expression. Expression data which is associated with a reliability measure below a predetermined threshold is considering missing, i.e., as if no measure of degree of expression is available for that particular piece of data.

Sometimes, it is possible to impute missing data if the measured degree of expression is supported by other experiments within the dataset and if the measure of reliability of the missing data is at least another predetermined threshold. Thus, with corroboration, a slightly less reliable measured expression is acceptable and is therefore not considered missing.

In this illustrative embodiment, system 100 makes two types of data imputation available to the user, who select one or the other to be applied to each of arrays 102 prior to processing in the manner described above. In particular, the user selects between the known K-nearest neighbor imputation mechanism, the known gene mean value imputation mechanism, or no data imputation at all. Other data imputation mechanisms can also be used. Effective and accurate data imputation significantly improves the accuracy of processing by system 100 since a greater number of samples are provided for statistical analysis in the manner described above.

Data filtering removes unreliable expression data from arrays 102. Unreliable expression data can erroneously influence statistical analysis by system 100. Accordingly, the user can specify effective checks on unreliable data.

First, the user can specify, using user interface 114 for example, a predetermined range of acceptable expression values. Any value outside that predetermined range is excluded as unreliable.

Second, the user can specify a predetermined minimum allowable difference between minimum and maximum expression values for a particular column of expression data. Accordingly, if an experiment has insufficient variance between the various expression values thereof, the experiment is considered unreliable and is removed from arrays 102. Accordingly, such unreliable expression data is not permitted to improperly influence statistical processing in the manner described above.

Inter-Dataset Mapping

It is sometimes desirable to use data from one dataset as a supervising array for a different dataset. Such is difficult, however, as experiments represented by experiment metadata 1004 (FIG. 10) is generally not sorted or otherwise organized in any particular sequence. Different datasets typically include different numbers of experiments and the experiments generally do not correspond to one another. Specifically, metadata stored in experiment metadata 1004 of one dataset generally does not correspond to similarly positioned metadata stored in experiment metadata of another dataset.

As a result, a row of expression data from one dataset cannot generally be used as a supervising array for another dataset. To make such inter-dataset analysis feasible, such a row of expression data can be mapped from one dataset to another.

Inter-dataset mapping between first and second datasets of class label, time series, and survival time supervising arrays is generally unnecessary. In particular, class labels are determined according to metadata associated with each experiment. Accordingly, the class labels of the second dataset are generated from the metadata of the second dataset and reference to the first dataset is unnecessary. Survival time supervising arrays are similarly generated from metadata of the experiments in question; mapping of a preexisting supervising array is therefore unnecessary. Time series supervising arrays are similarly derived from metadata of the experiments, and mapping of time series supervising arrays from one dataset to another is therefore similarly not necessary.

However, expression value supervising arrays rely on the relative positions of expression values corresponding to positions of analogous expression values in the array to be clustered or correlated in accordance with the supervising array. In particular, the expression arrays of FIGS. 10-12 are all accurately described by experiment metadata 1004 due to the analogous organization of expression data within those arrays. However, an expression value supervising array such as supervising array 1202 is not applicable to another dataset since the experiment metadata of that other dataset is most likely not accurately descriptive of supervising array 1202.

To apply a supervising array from one dataset to another, the supervising array must be mapped to the other dataset such that the metadata of the other dataset corresponds to the mapped supervising array. Such mapping of an expression value supervising array forms an equivalent expression value supervising array which corresponds to the experiment metadata of the second dataset. Thus, for each experiment of the second dataset, an expression value for the newly mapped supervising array must be determined.

Determining a mapped expression value for a particular experiment generally includes (i) reference to the experiment metadata of the particular experiment, (ii) mapping of experiment metadata of the first dataset to the experiment metadata of the second dataset, and (iii) selection of a new expression value according to that mapping.

In one illustrative embodiment, experiment metadata of both datasets includes a number of classes, e.g., various types of cancer and/or various stages of cancer of patients from which the experiments were taken. For illustration purposes, it is helpful to consider an example in which there are three (3) classes denoted by respective numerals, 0, 1, and 2. To map a supervising array to a new dataset, the class of each new expression value in a new, mapped supervising array is determined, and an expression value is selected according to the class. For example, if the first experiment of the new dataset has a class of 0, the first expression value of the new, mapped supervising vector is selected from one or more experiments of the original supervising array whose class is also 0. The expression value can be an average expression value of all experiments of the original supervising array whose class is 0, can be a randomly selected one of the experiments of the original supervising array whose class is 0, or can be selected some other way. Once each expression value of the new, mapped supervising array is selected, the new supervising array has been completely mapped.

When class labels aren't available or are not interesting to the user, new expression values are selected according to experiment metadata which is closest to the experiment metadata of the mapped experiment in question in the new dataset. The user can select one or more of the fields in the experiment metadata which are of interest. Alternatively, all fields of the experiment metadata can be used. Known and conventional correlation techniques can be used to correlate experiment metadata of the original dataset to the metadata of the experiment in question in the new dataset, using the latter metadata as a response variable. The resulting correlation model can then be used to derive an expression value from the original supervising array from the associated experiment metadata for the new, mapped supervising array.

The above description is illustrative only and is not limiting. Instead, the present invention is defined solely by the claims which follow and their full range of equivalents.

What is claimed is:

1. A method for processing a first collection of expression data, the method comprising:
forming one or more clusters of the first collection of expression data;
using a selected one or more of the clusters as at least one response variable for processing a second collection of expression data.

2. The method of claim 1 wherein the first and second collections of expression data are the same collection of expression data.

3. The method of claim 1 wherein processing the second collection of expression data comprises:
forming one or more clusters of the second collection of expression data.

4. The method of claim 1 wherein processing the second collection of expression data comprises:
analyzing correlation of the expression data of the second collection to the at least one response variable.

5. The method of claim 1 wherein the first and second collections of expression data include genetic expression data.

6. The method of claim 1 wherein the first and second collections of expression data include proteomic expression data.

7. A method for processing expression data, the method comprising:
forming a first collection of one or more clusters from a first collection of expression data;
forming a second collection of one or more clusters from a second collection of expression data;
correlating the first and second collection of clusters.

8. The method of claim 7 wherein correlating comprises:
using one or more selected clusters of the second collection of clusters as a response variable for correlation processing of the first collection of clusters.

9. The method of claim 7 wherein correlating comprises:
producing a correlation model by which one or more selected ones of the second collection of clusters can be predicted using one or more selected ones of the first collection of clusters.

10. The method of claim 7 wherein the first and second collections of expression data are the same collection of expression data.

11. The method of claim 7 wherein forming the first collection of clusters and forming the second collection of clusters are performing using a single cluster tool.

12. The method of claim 7 wherein the first and second collections of expression data include genetic expression data.

13. The method of claim 7 wherein the first and second collections of expression data include proteomic expression data.

14. A method for processing expression data, the method comprising:
analyzing correlation of a first collection of expression data to at least a first response variable to produce first correlation data;
analyzing correlation of a second collection of expression data to at least a second response variable to produce second correlation data; and
comparing the first and second correlation data.

15. A computer readable medium useful in association with a computer which includes a processor and a memory, the computer readable medium including computer instructions which are configured to cause the computer to process a first collection of expression data by:
forming one or more clusters of the first collection of expression data;
using a selected one or more of the clusters as at least one response variable for processing a second collection of expression data.

16. The computer readable medium of claim 15 wherein the first and second collections of expression data are the same collection of expression data.

17. The computer readable medium of claim 15 wherein processing the second collection of expression data comprises:
forming one or more clusters of the second collection of expression data.

18. The computer readable medium of claim 15 wherein processing the second collection of expression data comprises:
analyzing correlation of the expression data of the second collection to the at least one response variable.

19. The computer readable medium of claim 15 wherein the first and second collections of expression data include genetic expression data.

20. The computer readable medium of claim 15 wherein the first and second collections of expression data include proteomic expression data.

21. A computer readable medium useful in association with a computer which includes a processor and a memory, the computer readable medium including computer instructions which are configured to cause the computer to process expression data by:
forming a first collection of one or more clusters from a first collection of expression data;
forming a second collection of one or more clusters from a second collection of expression data;
correlating the first and second collection of clusters.

22. The computer readable medium of claim 21 wherein correlating comprises:
using one or more selected clusters of the second collection of clusters as a response variable for correlation processing of the first collection of clusters.

23. The computer readable medium of claim 21 wherein correlating comprises:
producing a correlation model by which one or more selected ones of the second collection of clusters can be predicted using one or more selected ones of the first collection of clusters.

24. The computer readable medium of claim 21 wherein the first and second collections of expression data are the same collection of expression data.

25. The computer readable medium of claim 21 wherein forming the first collection of clusters and forming the second collection of clusters are performing using a single cluster tool.

26. The computer readable medium of claim 21 wherein the first and second collections of expression data include genetic expression data.

27. The computer readable medium of claim 21 wherein the first and second collections of expression data include proteomic expression data.

28. A computer readable medium useful in association with a computer which includes a processor and a memory, the computer readable medium including computer instructions which are configured to cause the computer to process expression data by:
analyzing correlation of a first collection of expression data to at least a first response variable to produce first correlation data;
analyzing correlation of a second collection of expression data to at least a second response variable to produce second correlation data; and
comparing the first and second correlation data.

29. A computer system comprising:
a processor;
a memory operatively coupled to the processor; and
an expression data processing module (i) which executes in the processor from the memory and (ii) which, when executed by the processor, causes the computer to process a first collection of expression data by:
forming one or more clusters of the first collection of expression data;
using a selected one or more of the clusters as at least one response variable for processing a second collection of expression data.

30. The computer system of claim 29 wherein the first and second collections of expression data are the same collection of expression data.

31. The computer system of claim 29 wherein processing the second collection of expression data comprises:
forming one or more clusters of the second collection of expression data.

32. The computer system of claim 29 wherein processing the second collection of expression data comprises:
analyzing correlation of the expression data of the second collection to the at least one response variable.

33. The computer system of claim 29 wherein the first and second collections of expression data include genetic expression data.

34. The computer system of claim 29 wherein the first and second collections of expression data include proteomic expression data.

35. A computer system comprising:
a processor;
a memory operatively coupled to the processor; and
an expression data processing module (i) which executes in the processor from the memory and (ii) which, when executed by the processor, causes the computer to process a first collection of expression data by:
forming a first collection of one or more clusters from a first collection of expression data;
forming a second collection of one or more clusters from a second collection of expression data;
correlating the first and second collection of clusters.

36. The computer system of claim 35 wherein correlating comprises:
using one or more selected clusters of the second collection of clusters as a response variable for correlation processing of the first collection of clusters.

37. The computer system of claim 35 wherein correlating comprises:
producing a correlation model by which one or more selected ones of the second collection of clusters can be predicted using one or more selected ones of the first collection of clusters.

38. The computer system of claim 35 wherein the first and second collections of expression data are the same collection of expression data.

39. The computer system of claim 35 wherein forming the first collection of clusters and forming the second collection of clusters are performing using a single cluster tool.

40. The computer system of claim 35 wherein the first and second collections of expression data include genetic expression data.

41. The computer system of claim 35 wherein the first and second collections of expression data include proteomic expression data.

42. A computer system comprising:
a processor;
a memory operatively coupled to the processor; and
an expression data processing module (i) which executes in the processor from the memory and (ii) which, when executed by the processor, causes the computer to process a first collection of expression data by:
analyzing correlation of a first collection of expression data to at least a first response variable to produce first correlation data;
analyzing correlation of a second collection of expression data to at least a second response variable to produce second correlation data; and
comparing the first and second correlation data.
